(19) United States
(12) Patent Application Publication
(10) Pub. No.: US 2016/0200762 A1
(43) Pub. Date: Jul. 14, 2016

(54) Bioactive polypeptide DELQ and preparation method as well as application thereof

(71) Applicants: SHANGHAI JIAOTONG UNIVERSITY, Shanghai (CN); ZHEJIANG PANDA DAIRY GROUP COMPANY LIMITED, Wenzhou, Zhejiang (CN)

(72) Inventors: Shaohui Zhang, Shanghai (CN); Shanshan Lu, Shanghai (CN); Guanhua Sun, Shanghai (CN); Liuliu Ma, Shanghai (CN); Jiehui Zhou, Shanghai (CN); David Xi An Li, New South Wales (AU); Dongsheng Zhan, Shanghai (CN)

(73) Assignees: SHANGHAI JIAOTONG UNIVERSITY, Shanghai (CN); ZHEJIANG PANDA DAIRY GROUP COMPANY LIMITED, Wenzhou, Zhejiang (CN)

(21) Appl. No.: 14/434,094

(22) PCT Filed: Dec. 12, 2013
(86) PCT No.: PCT/CN2013/089196

§ 371 Date: Apr. 7, 2015

(30) Foreign Application Priority Data
Dec. 12, 2012 (CN) 201210536557.8

(51) Int. Cl. C07K 5/113; C12P 21/02

(52) U.S. Cl. CPC C07K 5/1021; C12P 21/02; A23L 1/3053; A23L 1/3056; A61K 38/00

(57) ABSTRACT

A milk-derived bioactive polypeptide has in vitro antioxidant activity and an immunity-enhancing function, and an amino acid sequence thereof is DELQ. According to in vitro antioxidant and in vitro immunity-enhancing experiments, it is proved that the peptide has good antioxidant activity and promotes immune system activity.

### CROSS REFERENCE OF RELATED APPLICATION

This is a U.S. National Stage under 35 U.S.C 371 of the International Application PCT/CN2013/089196, filed Dec. 12, 2013, which claims priority under 35 U.S.C. 119(a-d) to CN 201210536557.8, filed Dec. 12, 2012.

### BACKGROUND OF THE PRESENT INVENTION

1. Field of Invention

The present invention relates to a field of protein, and more particularly to a bioactive polypeptide DELQ and a preparation method as well as an application thereof.

2. Description of Related Arts

In a fermentation process of milk with lactic acid bacteria, some milk proteins are consumed by the lactic acid bacteria for metabolism, and a series of physiological and biochemical reactions will happen, in such a manner that the proteins are transformed into polypeptides or free amino acids, for being digested and absorbed by human body or being absorbed and transported directly into blood circulation through intestinal epithelial cells. Among the polypeptides, some have special physiological functions, and are called “bioactive peptide”.

Oxidation reaction and oxidative metabolism are essential for both food and human body. Radicals and active oxygen cause a series of oxidation reactions. When radical is overdose, protection of protective enzymes such as superoxide dismutase and catalase is insufficient, which results in a series of side-effects such as lipid oxidation and apoptosis. Such oxidation reactions not only affect the shelf life of foods containing fat, but also cause some harm on human health, such as rheumatoid arthritis, diabetes and arteriosclerosis. In addition, in 2005, Collins et al. found that the incidence of cancer is also related with oxidative damage to DNA.

Some early synthetic antioxidants such as butylated hydroxy anisole (BHA), 2,6-di-tert-butyl-4-methylphenol (BHT) are applied to food as a lipid antioxidant. However, the artificial synthetic additives have potential risks for humans. Therefore, to find safe antioxidants from natural food is important. In recent years, it was found that some food-derived polypeptide materials have good anti-oxidation, such as corn peptides, soy peptides and milk polypeptides. These polypeptides can be produced by microbial fermentation, enzymatic digestion, etc. Additionally, most of the polypeptides with antioxidant activity are formed by 2-20 amino acid residues, a molecular weight thereof is less than 6000 Da, and the polypeptide comprises a certain amount of hydrophobic amino acids and aromatic amino acids.

Since the discovery of opioid peptides, immune active polypeptide is the first type of bioactive peptide which is obtained from milk and whose physiological activity has been proved. In 1981, Jolles et al. first discovered that hexapeptide with an amino acid sequence of Val-Glu-Pro-Ile-Pro-Tyr is able to be obtained by hydrolyzing human milk protein with trypsase. In vitro experiments show that the hexapeptide is able to enhance phagocytosis of mouse peritoneal macrophages on sheep red blood cells. Migliore-Samour et al. found that hexapeptide, Thr-Thr-Met-Pro-Leu-Trp, derived from casein is able to stimulate phagocytosis of sheep red blood cells on mouse peritoneal macrophages and enhance resistance to Klebsiella pneumoniae. Li Suping et al. fed rats with synthetic milk-derived peptide (PGPIPN), and found that phagocytosis of rat peritoneal macrophages and immunomodulatory functions associated with red blood cells are significantly enhanced.

Research shows that the immune active peptides not only enhance immunity, stimulate proliferation of body lymphocyte, enhance phagocytosis of macrophage, promote release of cytokines, improve ability to withstand external pathogens, and reduce incidence of the body, but also cause no immune rejection of human body.

### SUMMARY OF THE PRESENT INVENTION

A first object of the present invention is to provide a bioactive polypeptide, wherein an amino acid sequence thereof is Asp-Glu-Leu-Gln (as shown in SEQ ID NO: 1).

Preferably, the bioactive polypeptide is milk-derived.

According to the present invention, the bioactive polypeptide DELQ is milk-derived, specifically from β-casein, and is Nos. 58-61 amino acid residues of the β-casein.

Preferably, the bioactive polypeptide has functions such as in vitro antioxidant activity and immunity-enhancing.

According to the present invention, the bioactive polypeptide is able to be manually prepared by genetic engineering methods, and is also able to be obtained from milk by separating and purifying.

According to the present invention, a nucleotide fragment of the bioactive polypeptide is encoded.

An amino acid sequence and a nucleotide sequence of the β-casein relate to conventional technologies. By encoding the nucleotide fragment of the Nos. 58-61 amino acid residues of the β-casein, the mature bioactive polypeptide DELQ is able to be encoded.

Furthermore, the nucleotide fragment of the bioactive polypeptide is encoded, and a sequence thereof is 5′-gatgaactccag-3′ (SEQ ID NO: 2).

A second object of the present invention is to provide a preparation method of the bioactive polypeptide, comprising steps of:

1) fermenting: adding Lactobacillus helveticus into skim milk for anaerobic fermentation, in such a manner that Lactobacillus helveticus fermented milk is obtained;

2) coarsely extracting the polypeptide: centrifugally separating the Lactobacillus helveticus fermented milk obtained in the step 1) with a low temperature, and collecting supernatant; and

3) purifying the polypeptide, comprising steps of:

a) ultrafiltering the supernatant obtained in the step 2), and collecting filtrate; and

b) separating the filtrate collected by reversed phase high-performance liquid chromatogram separation through a reversed phase chromatographic column SOURSE 5 RPC ST (4.6×150 mm), and collecting a bioactive polypeptide DELQ.

According to the present invention, the skim milk is milk which is skimmed. Usually, a fat content thereof is less than 0.1%.

Preferably, conditions of the anaerobic fermentation are: a fermentation temperature of 36-38° C., and a fermentation time of 15-20 h, preferably 19 h.

Preferably, in the step 2), low-temperature-centrifugation conditions are: a temperature of 4° C., a centrifugation rate of 8000-10000 rpm, and a centrifugation time of 15-30 min

Preferably, in the step a) of the step 3), molecular weight cut-offs of filter membranes for ultrafiltering are respectively 10 kDa and 3 kDa. According to the present invention, two filter membranes with the molecular weight cut-offs of 10 kDa and 3 kDa are utilized, and a sample passes the two filter membranes in sequence for ultrafiltering.

Preferably, in the step a) of the step 3), during ultrafiltering, a pressure range is 0.1-0.3 MPa, and a filtrate flow rate is 0.8-1.2 mL/min

Preferably, in the step b) of the step 3), during the reversed phase high-performance liquid chromatogram separation, a mobile phase A is ddH₂O comprising 2% acetonitrile and 0.05% TFA; a mobile phase B is 100% acetonitrile.

Preferably, in the step b) of the step 3), during the reversed phase high-performance liquid chromatogram separation, a polypeptide eluting peak with a molecular weight of 504.23 Da is collected, which is the bioactive polypeptide DELQ.

According to the present invention, during the reversed phase high-performance liquid chromatogram separation, a molecular weight of the DELQ is known. The polypeptide eluting peak with the molecular weight of 504.23 Da is collected, which is the bioactive polypeptide DELQ. Specifically, a retention time thereof is 24 min

A third object of the present invention is to provide a method for preparing antioxidant and/or immunity-enhancing foods, health products and medicines, comprising adding an appropriate dose of the bioactive polypeptide in the foods, health products and medicines.

According to the present invention, the bioactive polypeptide DELQ is applicable to prepare dairy products such as yoghurt, and cosmetics which are capable of reducing harm on skins caused by free radicals. Furthermore, because of being directly absorbed without degradation, the bioactive polypeptide DELQ is applicable to prepare the immunity-enhancing health products, or the antioxidant and/or immunity-enhancing medicines.

A fourth object of the present invention is to provide an antioxidant medicine, comprising a bioactive polypeptide DELQ or derivatives of the DELQ.

A fifth object of the present invention is to provide an immunity-enhancing medicine, comprising a bioactive polypeptide DELQ or derivatives of the DELQ.

The derivatives of the DELQ are derivatives obtained by modifying the polypeptide on amino acid side chain groups, amino ends or carboxyl ends by hydroxylation, carboxylation, carbonylation, methylation, acetylation, phosphorylation, esterification or glycosylation.

Advantages of the bioactive polypeptide DELQ are as follows. The milk-derived bioactive polypeptides DELQ according to the present invention have good antioxidant activity and promote immune system activity. On one hand, free radicals are reduced in the body for reducing harm caused by the free radicals on the human body; on the other hand, the bioactive polypeptide DELQ is also able to enhance immunity, enhance phagocytic function of macrophages, improve resistance abilities to external pathogens, and reduce incidence of the body. Furthermore, the bioactive polypeptide DELQ is able to be directly absorbed by gastrointestinal tract without degradation, and immune rejection will not be activated, which is quite important for development of dairies and health products with antioxidant and immune-enhancing functions.

### BRIEF DESCRIPTION OF THE DRAWINGS

FIG. 1 is a mass spectrometry comparison chart of crude extracts of Lactobacillus helveticus fermented skim milk and unfermented skim milk after ultrafiltering (wherein A: a mass spectrometry of the crude extract of the 3000 Da unfermented skim milk; and B: a mass spectrometry of the crude extract of the 3000 Da Lactobacillus helveticus fermented skim milk).

FIG. 2 illustrates molecular weight and abundance difference comparison of the crude extracts of the 3000 Da unfermented skim milk and the 3000 Da Lactobacillus helveticus fermented skim milk.

FIG. 3 is a reversed phase high-performance liquid chromatogram separation comparison chart of bioactive polypeptides of control fermented milk and Lactobacillus helveticus fermented milk (wherein curve-a: a reversed phase high-performance liquid chromatogram 215 nm elution map of the control fermented milk; and curve-b: a reversed phase high-performance liquid chromatogram 215 nm elution map of a 3000 Da supernatant of the Lactobacillus helveticus fermented milk.

FIG. 4 is a mass chromatography extraction chart (with m/z=504.23).

FIG. 5 is a level-1 mass chromatography of a fragment with a mass charge ratio of 504.23.

FIG. 6 is a level-2 mass chromatography of the fragment with the mass charge ratio of 504.23.

FIG. 7 illustrates az and by broken conditions as well as calculated sequences of the polypeptides with the mass charge ratio of 504.23.

FIG. 8 is a [DPPH.] methanol standard curve.

FIG. 9 illustrates a [DPPH.] radical removing rate of bioactive polypeptide from Lactobacillus helveticus fermented milk.

FIG. 10 is a FeSO₄ standard curve.

FIG. 11 is a total ion current chart of the DELQ

FIG. 12 is a level-1 mass chromatography of the DELQ.

### DETAILED DESCRIPTION OF THE PREFERRED EMBODIMENT

One skilled in the art will understand that embodiment of the present invention as shown in the drawings and described below is exemplary only and not intended to be limiting.

When numerical ranges are given in embodiments, it should be understood that, unless otherwise specified, any value at and between two endpoints of each numerical range can be selected. Unless defined otherwise, all technical and scientific terms used with the present invention are commonly understood by one skilled in art. In addition to the specific embodiment of the method, apparatus and materials in the examples, according to the prior art known to one skilled in the art and described in the present invention, similar or equivalent method, apparatus and materials are applicable.

Unless otherwise stated, the experimental methods, detection methods, and preparation methods of the present invention are all common in the art of conventional molecular biology, biochemistry, chromatin structure and analysis, analytical chemistry, cell culture, recombinant DNA technology and related fields. These techniques are well described in the literature in more details, see Sambrook et al., MOLECULAR CLONING: A LABORATORY MANUAL, Second edition, Cold Spring Harbor Laboratory Press, 1989 and Third edition, 2001; Ausubel et al., CURRENT PROTOCOLS IN MOLECULAR BIOLOGY, John Wiley & Sons, New York, 1987 and periodic updates; the series METHODS IN ENZYMOLOGY, Academic Press, San Diego; Wolffe, CHROMATIN STRUCTURE AND FUNCTION, Third edition, Academic Press, San Diego, 1998; METHODS IN ENZYMOLOGY, Vol. 304, Chromatin (PMWassarman and APWolffe, eds), Academic Press, San Diego, 1999; and METHODS IN MOLECULAR BIOLOGY, Vol. 1 19, Chromatin Protocols (PBBecker, ed) Humana Press, Totowa, 1999, etc.

### Example 1

### Preparation of Active Peptide

1. Preparation of Fermented Milk

1) Lactobacillus helveticus Fermented Milk

Preparing 12 wt % skim milk with skim milk powder (New Zealand NZMP skim milk powder) and water (which means adding 12 g skim milk powder in 88 g water, similarly below); under an aseptic condition, picking 3 loops of Lactobacillus helveticus bacterial colonies (CICC6024), adding to the sterilized 12 wt % skim milk, and thoroughly stirring; after seeding, sealing an opening with aluminum foil for preventing pollution; placing in a culture tank with a temperature of 37° C. for 19 h; after culturing, thoroughly stirring curdled milk for completing activation of the Lactobacillus helveticus and obtaining a leaven for Lactobacillus helveticus fermented milk.

Picking 10 mL prepared Lactobacillus helveticus leaven for being seeded to 500 mL sterilized 12 wt % skim milk (with a seeding rate of 2 v/v %), fermenting at 37° C. for 19 h, then thoroughly stirring curdled milk and storing at 4° C. for obtaining the Lactobacillus helveticus fermented milk.

2) Control Fermented Milk

Control fermented milk is prepared similarly with Lactobacillus Bulgaricus (LB340) and Streptococcus Thermophilus (TA40).

Specifically, under an aseptic condition, respectively picking 3 loops of Lactobacillus Bulgaricu bacterial colonies and 3 loops of Streptococcus Thermophilus bacterial colonies, adding to the sterilized 12 wt % skim milk, and thoroughly stirring; after seeding, sealing an opening with aluminum foil for preventing pollution; placing in a culture tank with a temperature of 37° C. for 19 h; after culturing, thoroughly stirring curdled milk for completing activation of the Lactobacillus Bulgaricus and the Streptococcus Thermophilus, and obtaining two leavens for preparing the control fermented milk.

Respectively picking 5 mL prepared Lactobacillus Bulgaricus leaven and 5 mL prepared Streptococcus Thermophilus leaven for being seeded to 500 mL sterilized 12 wt % skim milk (with a seeding rate of 2 v/v %), fermenting at 37° C. for 19 h, then thoroughly stirring curdled milk and storing at 4° C. for obtaining the control fermented milk.

2. Identification of Bioactive Polypeptide

I) Experiment Method

1) Sample Treatment

Respectively placing the Lactobacillus helveticus fermented milk, the control fermented milk and 12 wt % skim milk in centrifuge tubes for low-temperature-centrifugation, wherein low-temperature-centrifugation conditions are: a centrifugation rate of 9000 rpm, a temperature of 4° C., and a centrifugation time of 20 min; after centrifugation, filtering out deposition and collecting supernatants.

Respectively pouring the supernatants into ultrafiltering cups; for preventing the bioactive polypeptide from oxidation, opening a pressure valve of a nitrogen tank for inputting nitrogen; at the same time, starting a magnetic stirring device for preventing a concentration polarization phenomenon; passing the samples through 10 kDa and 3 kDa filter membranes, and collecting outputted filtrate.

During ultrafiltering, a flow rate is kept stable and the filtrate is kept clear. The flow rate is kept about 1 mL/min, and the pressure is 0.1-0.3 MPa. The filtrate of the Lactobacillus helveticus fermented milk, the control fermented milk and the 12 wt % skim milk is respectively collected as an experimental sample, a control sample and a blank sample, and is freeze-stored at −4° C.

2) Mass Spectrometric Analysis

Providing mass spectrometric analysis to the filtrate of the ultrafiltered Lactobacillus helveticus fermented milk (the experimental sample) and the ultrafilterred skim milk (the blank sample), mass spectrometric conditions thereof are:

Ion mode: ES+

Mass range (m/z): 100-1500

Capillary voltage (kV): 3.0

Sampling cone (V): 35.0

Ion source temperature (° C.): 100

Solvent-removing temperature (° C.): 350

Solvent-removing gas flow (L/hr): 600.0

Collision energy (eV): 6.0

Scanning time (sec): 0.3

Inner scanning time (sec): 0.02

II) Experiment Result

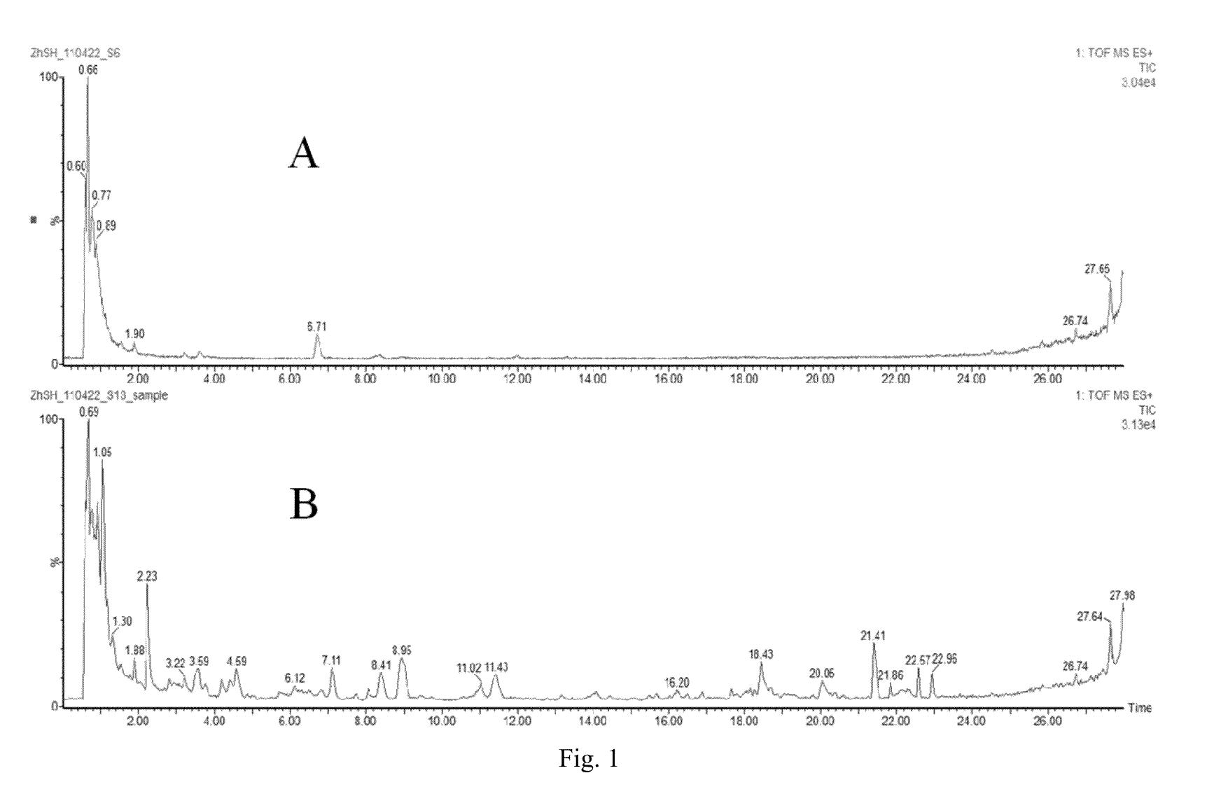

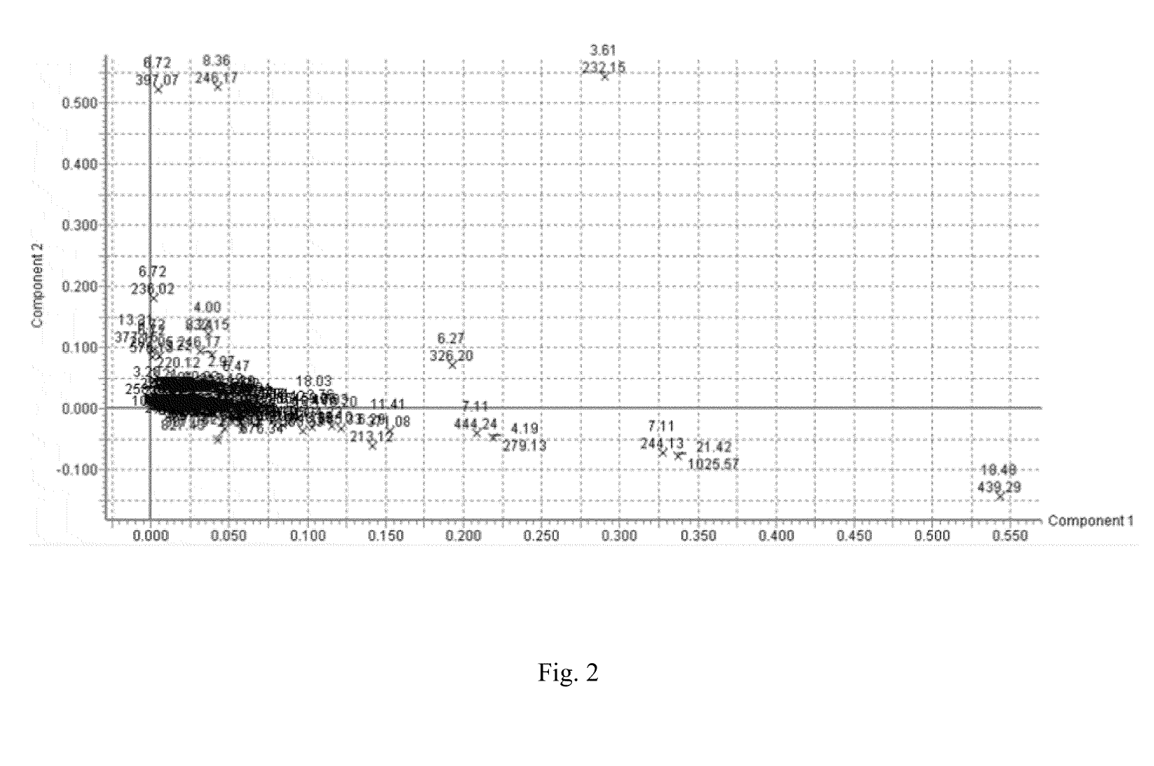

Mass spectrometric results of the filtrate of the ultrafiltered Lactobacillus helveticus fermented milk (the experimental sample) and that of the ultrafilterred skim milk (the blank sample) are illustrated in FIGS. 1-2. Referring to FIG. 1, a curve-A illustrates crude extract of 3000 Da unfermented skim milk (the blank sample); and a curve-B illustrates crude extract of 3000 Da Lactobacillus helveticus fermented skim milk (the experimental sample). Accordingly, after being fermented with the Lactobacillus helveticus, the crude extract of 3000 Da unfermented skim milk and the crude extract of 3000 Da Lactobacillus helveticus fermented skim milk change a lot in components. The retention times of the components are different due to different hydrophobicities. A mass range of the mass spectrometric is 100 Da-1500 Da. Therefore, it is known that with the skim milk below 1500 Da, small molecular substances absorbed are less at 210 nm. After fermented with Lactobacillus helveticus, substances with the molecular weight are efficiently increased, which illustrates that the polypeptide does not exist in unfermented skim milk, but in Lactobacillus helveticus fermented skim milk. It is also noticed that by increasing a fermentation time, an abundance of the polypeptide is efficiently increased, which further proves that by fermentation with the Lactobacillus helveticus, original large molecule proteins in the skim milk are resolved, and are transformed from the single large molecule protein into a large amount of complex small molecule polypeptides.

FIG. 2 illustrates abundance difference comparison results according to different molecular weights of the crude extracts of the 3000 Da unfermented skim milk (blank sample) and the 3000 Da Lactobacillus helveticus fermented skim milk (experimental sample). A longitudinal axis shows a molecular weight of substances in the crude extract of the skim milk, and a horizontal axis shows a molecular weight of substances in the crude extract of the fermented milk. Referring to the comparison, molecular weights of the substances in the Lactobacillus helveticus fermented milk and the unfermented skimmed milk with great differences caused by fermentation are obtained, thereby selecting parent ions with such mass charge ratios for being further analyzed by level-2 mass spectrometry.

Referring to FIG. 1, a content of a substance with a molecular weight of 397.07 Da and a retention time of 6.72 min is high in the crude extract of the skim milk substance (according to a peak of a map A of FIG. 1), which almost does not exist in the fermented milk. However, contents of a substance with a molecular weight of 232.15 Da and a retention time of 3.61 min are high in both the fermented milk and the skim milk. Therefore, components which are rich in the fermented milk and rare in the unfermented skim milk are compared, and comparison results are shown in Table 1.

TABLE 1
difference comparison of 3000Da crude extract of Lactobacillus helveticus
fermented milk and 3000Da crude extract of skim milk

| | | 3000Da crude | 3000Da crude |
|---|---|---|---|
| molecular | | extract of | extract of |
| weight (Da) | significance | skim milk (unit) | fermented milk (unit) |
| 504.2302 | 0.0779 | 0 | 37.23 ± 2.78 |
| 444.2442 | 0.1498 | 0 | 71.58 ± 4.27 |
| 504.2351 | 0.0886 | 0 | 41.72 ± 4.02 |
| 748.3877 | 0.0753 | 0 | 35.25 ± 2.46 |
| 439.291 | 0.3968 | 0 | 186.95 ± 10.67 |
| 1025.5662 | 0.2441 | 0 | 115.86 ± 7.17 |

According to MarkerLynx software analysis, molecular weight fragments with significant difference (p>0.05) are shown in Table 1. According to the abundance and the mass charge ratio, the polypeptide with 504.2302 Da and a retention time of 24 min are selected for level-2 mass spectrometry sequencing analysis.

3. Separating-Purifying of the Bioactive Polypeptide and Yield Comparison

I) Instruments and Reagents

Instruments: ÄKTA protein purification instrument purifier 10

Column specification: SOURSE 5 RPC ST4.6/150

Flow rate: 1 mL/min

Temperature: 25° C.

UV detection wavelength: 215 nm

Mobile phase A: ddH₂O comprising 2% acetonitrile and 0.05% TFA

Mobile phase B: 100% acetonitrile

Inputting volume: 1 mL

Gradient condition: 100% A at 0-7.5 min; 0% B to 50% B at 7.5-42.5 min; 50% B to 100% B at 42.5-45 min; 100% B at 45-50 min; and 0% A to 100% A at 50-72 min

II) Experiment Method

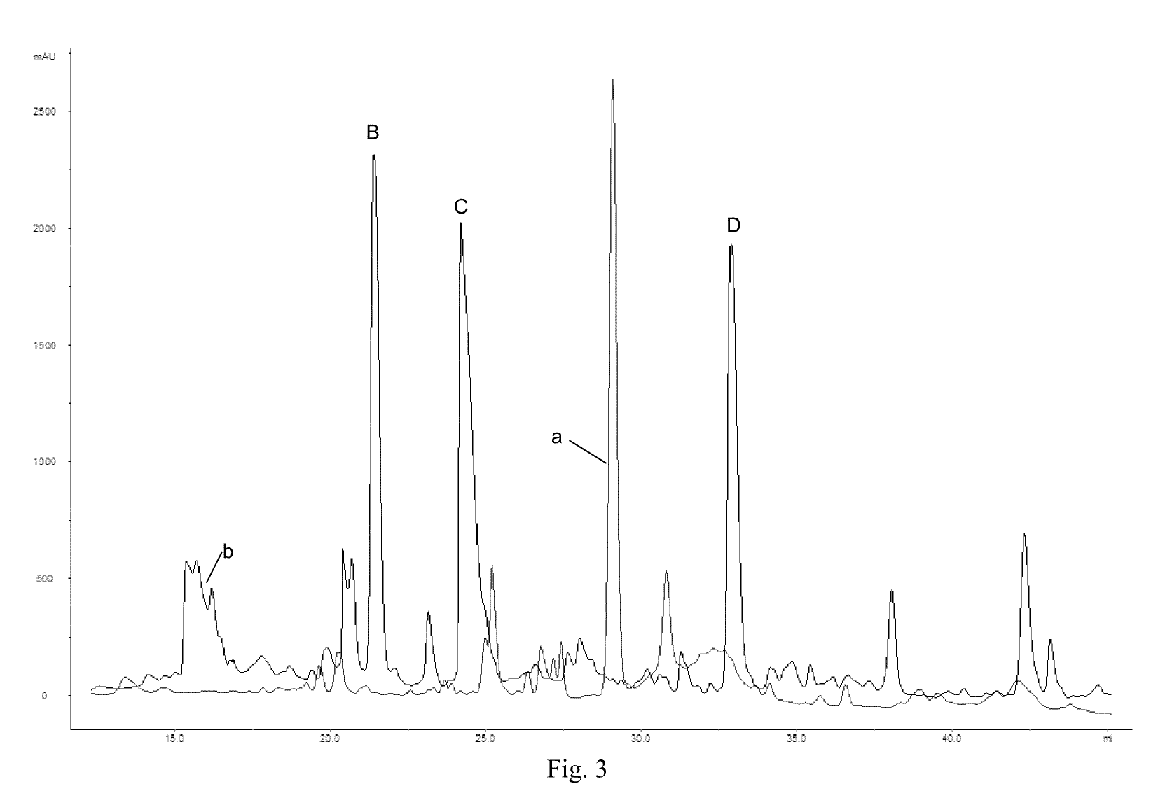

Pre-treatment of samples: diluting the experimental sample and the control sample with the mobile phase A (with a volume ratio of 1:1) as loading sample; processing the loading sample with reversed phase high-performance liquid chromatogram analysis, wherein results are shown in FIG. 3.

III) Experiment Results

Referring to FIG. 3, a curve-a is a reversed phase high-performance liquid chromatogram 215 nm elution map of the control fermented milk. A distinct absorption peak is at an elution time of 26 min, and other peaks are relatively low. According to a proportional relationship of absorption and a peptide concentration, it is considered that in the 12% control fermented milk 3000 Da supernatant (the control sample), less polypeptides exists, and specie is single. A curve-b is a reversed phase high-performance liquid chromatogram 215 nm elution map of a 3000 Da supernatant (the experimental sample) of the Lactobacillus helveticus fermented milk. Compared with the control fermented milk, absorption peaks in the reversed phase map of the 3000 Da supernatant of the Lactobacillus helveticus fermented milk are significantly increased, and 3 distinct absorption peaks exist at the elution times of 21 min, 24 min and 33 min During the experiment, the three peaks were collected, and respectively recorded as fermented milk isolate peaks B, C and D.

According to retention time comparison of the polypeptides corresponding to different molecular weights and molecular weight substances corresponding to original fermented milk isolate peaks B, C and D, it is found that 504.23 Da substances are derived from the fermented milk isolate peak C.

Referring to the comparison of the control fermented milk and the Lactobacillus helveticus fermented milk, it is found that milk fermented with the Lactobacillus helveticus comprises more the polypeptides with the molecular weight less than 3000 Da than the control fermented milk. The polypeptides are formed by polypeptide fragments and free amino acid released by resolving the original skim milk large proteins with intracellular enzymes and extracellular enzymes secreted by the Lactobacillus helveticus. The extracellular enzymes secreted by lactic acid have a non-specific or specific cut effect on dairy β-casein fragments. Usually, the polypeptides obtained by fermentation of microorganism have biological activity. If the Lactobacillus Bulgaricus and the Streptococcus Thermophilus are utilized for producing ordinary yogurt, the biological activity is relatively low due to a low production of polypeptide and the single species thereof

According to a principle of the reversed phase high-performance liquid chromatography, substances with poor hydrophobicity are firstly eluted and separated from a separation column due to a weak solid phase binding force with the separation column, while substance with good hydrophobicity are eluted and separated later from the separation column due to a good linkage with the separation column Therefore, hydrophobicity of the three isolates are ranked as follows: the Lactobacillus helveticus fermented milk isolate peak B>C>D. After collection, a sample of the peak C is obtained by a vacuum freeze-drying technology, and is stored at −4° C. as an experiment material for following mass spectrometric analysis and in vitro functional testing.

4. Quality of the Bioactive Polypeptides and Determination of an Amino Acid Sequence

I) Experimental Method

(1) Chromatographic Conditions:

Instrument: Waters ACQUITY UPLC ultra-performance liquid-electrospray-quadrupole-flight time mass analyzer

Column specification: BEH C18 column

Flow rate: 0.4 mL/min

Temperature: 45° C.

UV detection wavelength: 210 nm

Inputting volume: 7 μL

Gradient conditions: 99% A and 1% B at 0-3 min; 1% B to 5% B and 99% A to 95% A at 3-9 min; 5% B to 10% B and 95% A to 90% A at 9-15 min; 10% B to 25% B and 90% A to 75% A at 15-21 min; 25% B to 40% B and 75% A to 60% A at 21-24 min; 40% B to 80% B and 60% A to 20% A at 24-27 min; 80% B and 20% A at 27-27.5 min; 80% B to 5% B and 20% A to 95% A at 27.5 min-28 min; 5% B to 1% B and 95% A to 99% A at 28-28.5 min; and 99% A and 1% B at 28.5 min-30 min A: ddH₂O comprising 2% acetonitrile and 0.05% TFA; B: 100% acetonitrile.

(2) Mass Spectrometry Condition:

Ion mode: ES+

Mass range (m/z): 100-1500

Capillary voltage (kV): 3.0

Sampling cone (V): 35.0

Ion source temperature (° C.): 100

Solvent-removing temperature (° C.): 350

Solvent-removing gas flow (L/hr): 600.0

Collision energy (eV): 6.0

Scanning time (sec): 0.3

Inner scanning time (sec): 0.02

Level-2 mass spectrometry parent ion weight (m/z): 439.3

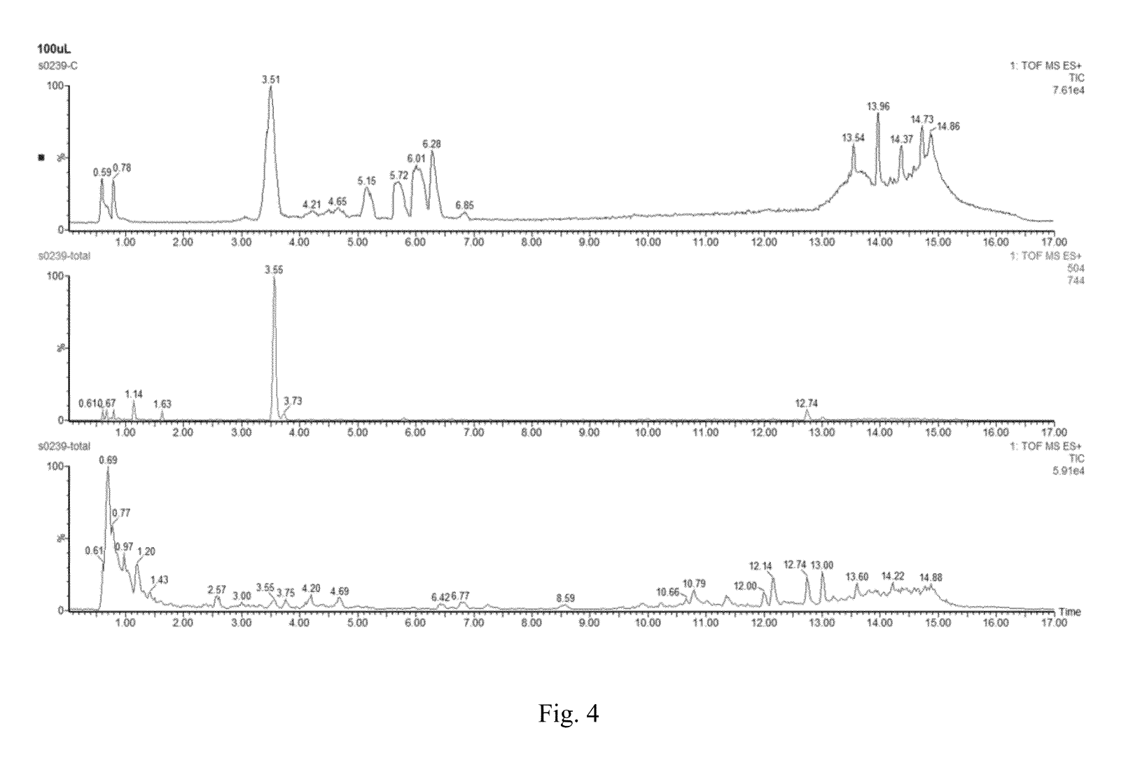

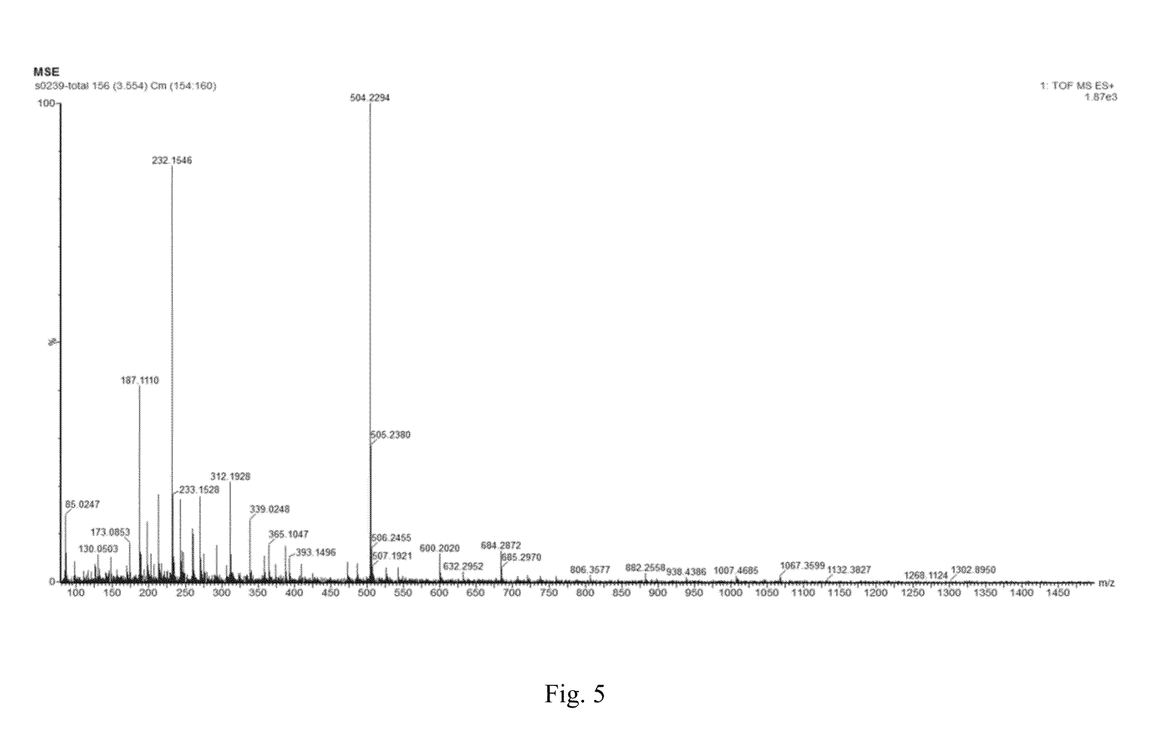

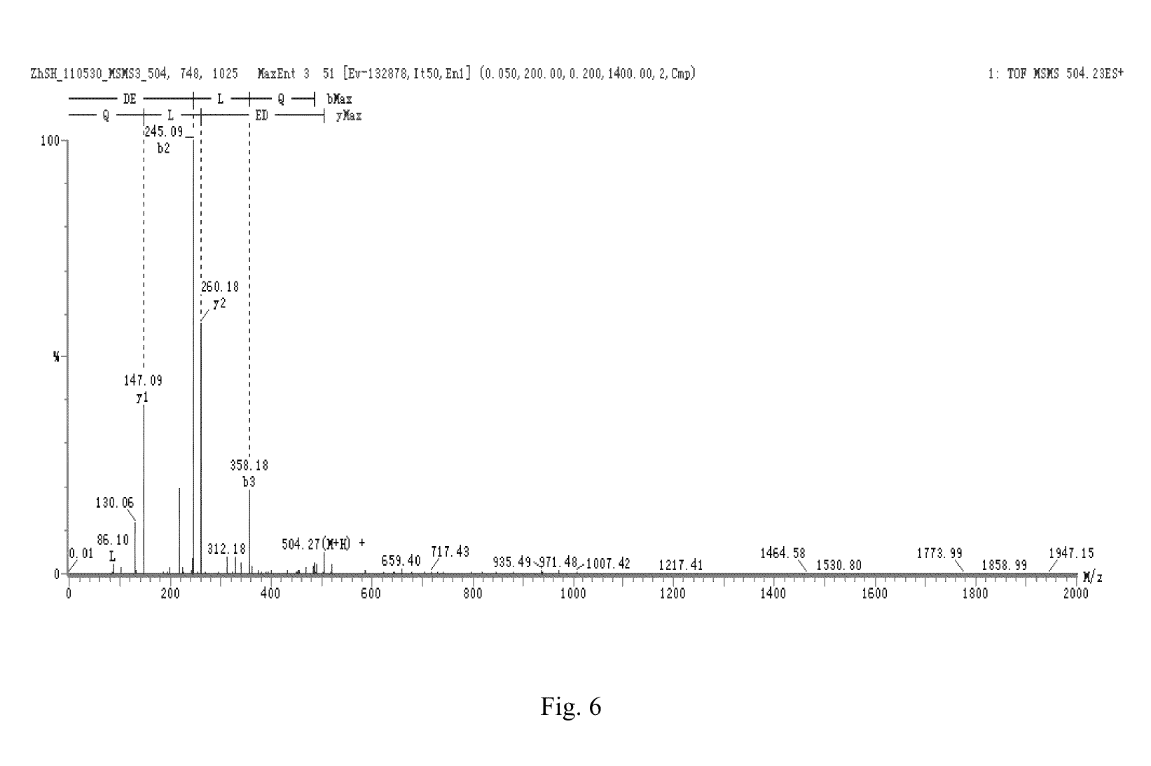

According to the experiment conditions, by the ultra-performance liquid-electrospray-quadrupole-flight time mass analyzer, a mass chromatography extraction chart, a level-1 mass chromatography and a level-2 mass chromatography of the 504.23 Da polypeptides at the Lactobacillus helveticus fermented milk isolate peak C are obtained, and the amino acid sequences are calculated by the Masslynx software, wherein results thereof are shown in FIGS. 4-7.

II) Experiment Result

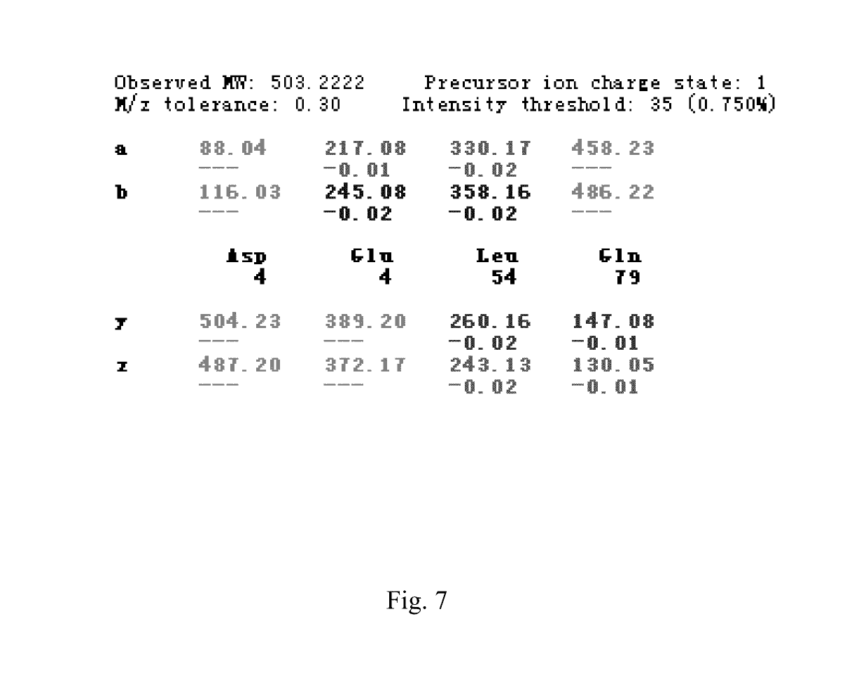

Referring to FIG. 7, according to az and by broken conditions and Masslynx software analysis, it is obtained that a sequence of the fragment with the mass charge ratio of 504.23 Da is Asp-Glu-Leu-Gln (DELQ), and marked as SEQ ID NO: 1. The fragment is derived from the Lactobacillus helveticus fermented milk isolate peak C, and is corresponding to Nos. 58-61 of β-casein residue sequences, wherein a β-casein amino acid sequence is GenBank No. AAA30431.1, the sequence is illustrated as SEQ ID NO: 3.

### Example 2

### Antioxidant Activity Experiment of Bioactive Peptide

Testing antioxidant activity of the bioactive polypeptide DELQ obtained in the Example 1 with a [DPPH.] (diphenyl picryl hydrazinyl radical) method and a FRAP (Ferric Reducing Ability Power) method.

I) Bioactive Polypeptide DELQ In Vitro Antioxidant Activity Detection with the [DPPH.] Method

1) Experiment Reagent and Instrument

Reagent: 1,1-Diphenyl-2-picrylhydrazyl [DPPH.], from Japan Wako company; methanol, from Shanghai traditional Chinese medicine company; and DELQ (C peak sample) prepared by fermenting Lactobacillus helveticus, obtained in the Example 1.

Main instrument: Sunrise microplate reader, from Austria Tecan company; 96-hole cell culturing plate, from U.S. Millipore company; and analytical balance, from Meitelei-tolido company.

2) Experimental Method

(1) Preparing 1 mmol/L [DPPH.] Methanol Solution:

Weighting 0.349 mg [DPPH.] with the analytical balance and dissolving in 1 ml methanol solution for obtaining 1 mmol/L [DPPF.] methanol solution, storing with tinfoil for avoiding light, wherein the [DPPF.] methanol solution is prepared before use.

(2) Standard Curve Detection of [DPPH.] Methanol Solution:

Adding 100 μL [DPPH.] methanol standard curve sample into the 96-hole cell culturing plate according to a Table 2, waiting for 90 min at a room temperature, detecting absorbance values at 517 nm with the microplate reader.

TABLE 2
preparation of [DPPH.]
methanol standard curve solution

| | 1 | 2 | 3 | 4 | 5 | 6 |
|---|---|---|---|---|---|---|
| [DPPH.] methanol | 100 | 80 | 60 | 40 | 20 | 0 |
| (μL) | | | | | | |
| methanol (μL) | 0 | 20 | 40 | 60 | 80 | 100 |
| [DPPH.] methanol | 1.0 | 0.8 | 0.6 | 0.4 | 0.2 | 0 |
| standard solution | | | | | | |
| (mmol/l) | | | | | | |

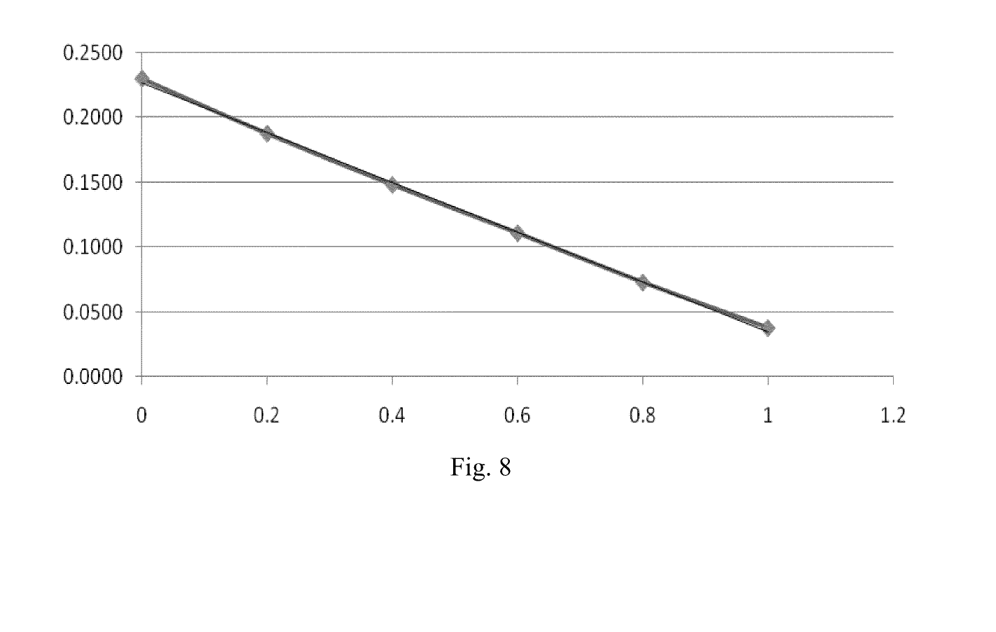

Accordingly, curves are fitted and a regression equation is calculated with Excel, and results thereof are shown in FIG. 8 (the regression equation: y=−0.192x+0.2271, R²=0.9991). A linear relationship of the [DPPH.] methanol standard curve is good, and a correlation coefficient thereof is 0.999, indicating that accuracy and precision of the [DPPH.] methanol standard curve satisfies testing requirements. From the results, absorbance values and [DPPF.] contents have an inverse relationship, i.e. the less the [DPPH.] content is, the higher the absorbance value will be, which means a stronger radical removing ability.

(3) Antioxidant Activity Detection of the Bioactive Polypeptide DELQ with [DPPH.] Method

1) sample group: adding 80 μL 1 mmol/L [DPPH.] methanol solution into the 96-hole cell culturing plate, and respectively adding 20 μL test sample (DELQ), positive control 1 (2.5 mg/mL Trolox), positive control 2 (0.025 mg/mL Trolox), and negative control (phytic acid) with different concentrations according to Table 3; and

2) blank group: adding 80 μL 1 mmol/L [DPPF.] methanol solution and 20 μL deionized water into the same 96-hole cell culturing plate for forming a blank group.

After adding the test sample, waiting at the room temperature for 90 min, detecting the absorbance values at 517 nm with the microplate reader, and calculating a radical removing ratio according to a following formula, wherein results thereof are shown in Table 3.

$$[ DPPH \cdot ] radical removing ratio = \frac{OD blank - OD sample}{OD blank} \times 100 \% \qquad Formula$$

TABLE 3
antioxidant activity detection result of bioactive polypeptide in
Lactobacillus helveticus fermented milk by [DPPH.] method

| | sample concentration (mg/mL) | | | | | |
|---|---|---|---|---|---|---|
| sample name | | 10.00 | 5.00 | 2.50 | 1.25 | 0.625 |
| test sample | | 24.98 ± 0.05 | 22.76 ± 0.01 | 19.04 ± 0.01 | 16.64 ± 0.01 | 2.28 ± 0.12 |
| (DELQ) | | | | | | |
| positive control 1 | 99.96 ± | | | | | |
| (2.5 mg/mL Trolox) | 0.0016 | | | | | |
| positive control 2 | 71.08 ± 0.03 | | | | | |
| (0.025 g/mL | | | | | | |
| Trolox) | | | | | | |
| negative control | 58.49 ± 0.08 | | | | | |
| (phytic acid) | | | | | | |

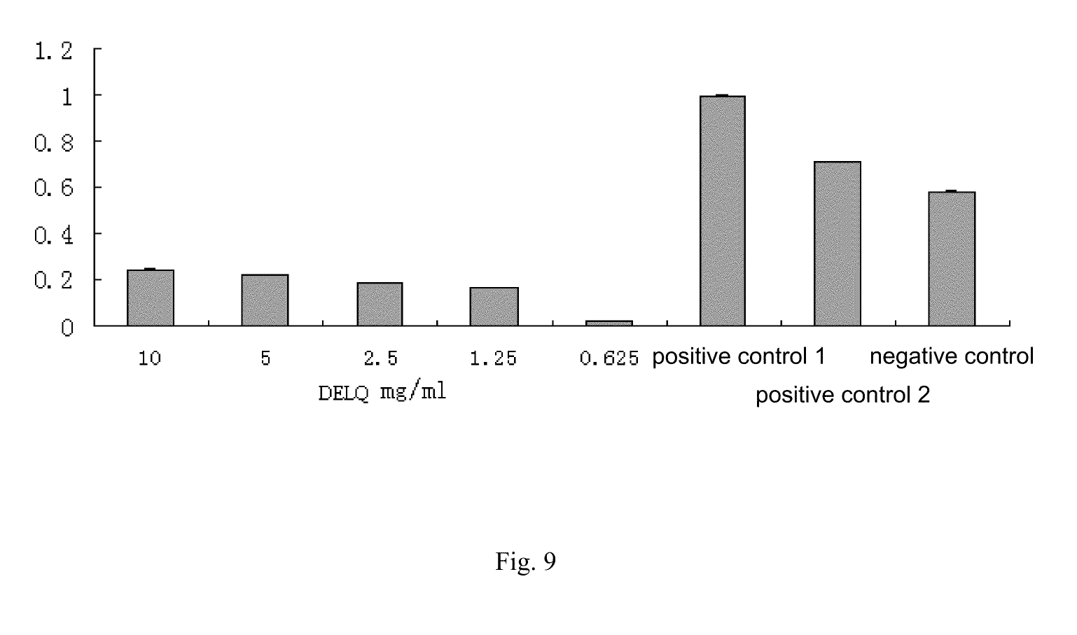

Referring to FIG. 9, under same conditions, the positive control 1 with 2.5 mg/mL Trolox has a strongest radical-removing ability, which is able to remove almost all the radicals in the solution, and followed by the 0.025 mg/mL Trolox, the phytic acid and the isolated peptide of the C peak of fermented milk isolate. A [DPPH.] radical-removing rate of the isolated peptide of the C peak of the fermented milk isolate is 24.98%, and decreases while the DELQ concentration decreases.

II) In Vitro Antioxidant Activity Detection of the Polypeptide in the Fermented Milk with the FRAP Method
1) Reagent and Instrument

FRAP detection reagent kit, from Shanghai Biyuntian biotechnology company; FeSO₄ solution (10 mmol/L), water-soluble vitamin E (Trolox solution) (10 mmol/L), the milk-derived bioactive polypeptide DELQ prepared by fermenting Lactobacillus helveticus obtained in the Example 1.

Main instrument: Sunrise microplate reader, from Austria Tecan company; 96-hole cell culturing plate, from U.S. Millipore company; analytical balance, from Meitelei-tolido company; and HWS26 electric thermostatic water bath, from Shanghai Infineon Technologies Limited.

2) Experimental Methods

(1) Preparing FRAP Working Fluid:

According to the FRAP detection reagent kit, thorough mixing 7.5 mL TPTZ diluent, 750 μL TPTZ solution, and 750 μL detection buffer, incubating in a 37° C. water bath, and using in 2 h.

(2) Drawing a FeSO₄ Standard Curve:

Adding 180 μL FRAP working fluid into the 96-hole cell culturing plate, then adding 5 μL FeSO₄ standard curve solution according to Table 4, gently mixing, incubating at 37° C. for 3-5 min, and detecting absorbance values at 593 nm with the microplate reader.

TABLE 4
solution preparation of FeSO₄
standard curve detection

| | 1 | 2 | 3 | 4 | 5 | 6 |
|---|---|---|---|---|---|---|
| FeSO₄ solution | 10 | 5 | 2 | 1 | 0.5 | 0 |
| (μL) | | | | | | |
| ddH₂O | 0 | 5 | 8 | 9 | 9.5 | 10 |
| FeSO₄ standard | 1 | 0.5 | 0.2 | 0.1 | 0.05 | 0 |
| solution | | | | | | |
| (mmol/L) | | | | | | |

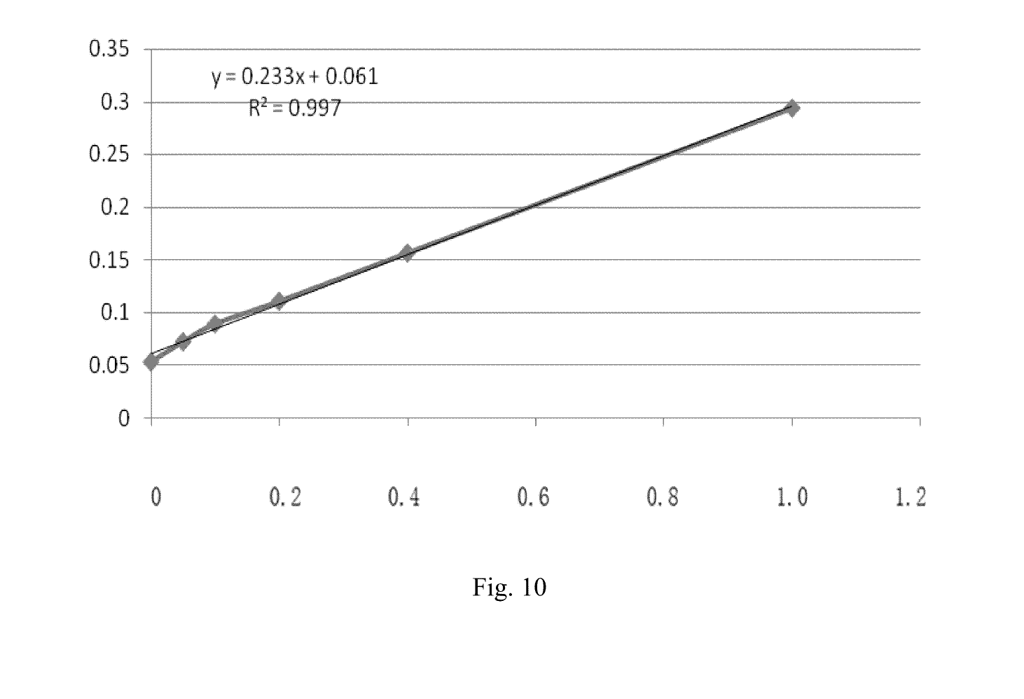

FeSO₄ concentration and the absorbance value have a good proportional relationship, i.e. the higher the FeSO₄ concentration is, the higher absorbance value will be. According to the present invention, FeSO₄ standard curve results are shown in FIG. 10, the standard curve has a good linear relationship, wherein a correlation coefficient thereof is 0.998. Precision and accuracy of the FeSO₄ curve satisfy testing requirements, and are applicable for subsequent calculation.

(3) Bioactive Polypeptide DELQ Antioxidant Capacity Detection with FRAP Method

Adding 180 μL FRAP working fluid into the 96-hole cell culturing plate, adding 5 μL ddH₂O into a blank control hole, adding 5 μL testing sample into a sample hole, adding 5 μL phytic acid into a positive control, gently mixing, incubating at 37° C. for 3-5 min, and detecting absorbance values at 593 nm with the microplate reader. FRAP is represented according to a concentration of the FeSO₄ standard solution. The radical-removing rate is calculated in accordance with a following formula, and results are shown in Table 5.

$$FRAP ( mmol \text{/} g ) = \frac{\begin{matrix}{FeSO}_{4} standard solution concentration \\ equaling to sample OD value ( mmol \text{/} g )\end{matrix}}{sample concentration ( m g \text{/} mL )}$$

TABLE 5
total antioxidant detection result of bioactive polypeptide
in Lactobacillus helveticus fermented milk by FRAP method

| | | | corresponding | |
|---|---|---|---|---|
| | | concen- | FeSO₄ | |
| | | tration | concentration | FRAP |
| | sample name | (mg/mL) | (mmol/L) | (mmol/g) |
| sample | polypeptide | 4.00 | 0.0835 ± 0.0351 | 0.0208 |
| group | DELQ | | | |
| positive | phytic acid | 4.00 | 0.0356 ± 0.0055 | 0.0089 |
| control group | | | | |

The in vitro total antioxidant activity of the polypeptide isolated from the Lactobacillus helveticus fermented milk is detected by the FRAP method. It is found that the DELQ in the Lactobacillus helveticus fermented milk isolate has a sufficient ability of restoring oxidizing substances. The FRAP thereof is 0.0208 mmol/g, which illustrates that the FRAP of the DELQ is higher than the FRAP of the phytic acid having the weak antioxidant activity under the same concentration. A significant difference exists (p>0.05). Therefore, the DELQ is considered to have significant antioxidant capacity.

### Example 3

### Promoting Immunity Activity Experiment of Bioactive Polypeptide

1. DELQ In Vitro Lymphocyte Proliferation Detection Experiment by MTT Method

1) Experiment Material and Instrument:

Reagent and material: experimental animal balb/c mouse (6-8 weeks old male, Shanghai Jiaotong University Agriculture and Biology College, Experimental Animal Center); the milk-derived bioactive polypeptide DELQ prepared by fermenting Lactobacillus helveticus; mouse lymphocyte extract (from Solarbio company); RPMI1640 medium (from GIBCO company); 3-(4, 5-dimethyl-2)-2, 5-diphenyl tetrazolium bromide (MTT for short, from Amresco company); concanavalin A (ConA for short, from Sigma company); bovine serum albumin (BSA for short, from Genebase company); pepsin (from Sigma company); trypsin (Corolase PP, from AB company).

Instrument: LRH-250F incubator, from Shanghai Yiheng Technology Co. Ltd; GL-22M high-speed refrigerated centrifuge, from Shanghai Hailu Xiangyi Centrifuge Instrument Co., Ltd.; Hera cell 150 CO₂ incubator, from Heraeus company; Dragon Wellscan MK3 microplate reader, from Labsystems company; ALPHA 1-2-LD vacuum freeze-drying machine, from Christ company; ultra-performance liquid chromatography-quadrupole-flight time mass analyzer, from waters company.

2) Experiment Method:

Obtaining a mouse spleen under an aseptic condition, and obtaining mouse lymphocytes with lymphocyte extracting liquid, for primary culture; adjusting a cell density to 2.5×10⁶ unit/mL with a RPMI1640 complete medium; adding 100 μL mouse lymphocyte suspension, 100 μL RPMI1640 complete medium, 20 μL concanavalin and 100 μL sample in sequence into the 96-hole cell culturing plate; wherein a blank control group (pH7.2-7.4, 3 mol/L PBS) and a negative control group (500 μg/mL BSA) are provided, which have been experimentally proved to have no effect on in vitro lymphocyte proliferation, and each group comprises three parallel experiment samples; culturing in 5% CO₂ in a 37° C. incubator for 68 h, adding 20 μL MTT to each hole under the aseptic condition, culturing for 4 h, carefully removing supernatant, adding 100 μL dimethyl sulfoxide to each hole, incubating at the 37° C. incubator for 10 min, thoroughly shaking, and detecting absorbance values at 570 nm with the microplate reader.

In vitro lymphocyte proliferation is represented as a stimulation index, and a calculation formula is as follows:

$$stimulation index = \frac{{A}_{3} - {A}_{1}}{{A}_{2} - {A}_{1}} \times 100 \%$$

wherein: A₁ is the absorbance value of the blank control group at 570 nm; A₂ is the absorbance value of the negative control group at 570 nm, and A₃ is the absorbance value of the experiment group at 570 nm.

3) Experiment Result and Analysis

TABLE 6
effect of bioactive polypeptide DELQ
on in vitro lymphocyte proliferation

| group | stimulation index SI |
|---|---|
| negative control group | 1 |
| DELQ | 1.154 ± 0.376* |

Note:
*represents comparison with negative control, wherein significant difference (P < 0.05) exists.

Experiment results are shown in Table 6. Referring to Table 6, when the concentration of the bioactive polypeptide is 100 μg/mL, the stimulation index of the DELQ is more than BSA, which illustrates that the DELQ promotes lymphocyte proliferation, wherein the stimulation index is up to 1.154 and the significant difference (P<0.05) exists between the negative control group and the DELQ. Therefore, it is identified that the DELQ isolated from the Lactobacillus helveticus fermented milk significantly promotes lymphocyte proliferation in mice. As a health product or additives, the DELQ is able to improve animal and human immunity.

### Example 4

### Experiment of Simulating Gastrointestinal Digestion on Bioactive Polypeptide DELQ

I) Simulating In Vitro Gastrointestinal Digestion

The experiment is divided into two steps. Firstly, preparing a 500 μg/mL DELQ solution, adding pepsin with a ratio of 20 mg the pepsin for every gram of the DELQ, adjusting a pH value of the reaction solution to 2.0, reacting with a temperature kept in a 37° C. thermostatic waterbath for 90 min; then adjusting a pH value to 7.5, and adding pancreatin with a ratio of 40 mg the pancreatin for every gram of the DELQ, and reacting with a temperature kept in a 37° C. thermostatic waterbath for 150 min; inactivating disgestive enzymes with a 95° C. waterbath for 5 min; and freeze-drying the reaction solution for obtaining dry powder thereof, and storing at −20° C. for further use.

II) Quality of Enzymic Hydrolysate and Amino Acid Sequence Determination

Picking 0.2 mg powder sample after stimulated in vitro gastrointestinal digestion, adding 50 μL water and 450 μL pure ethanol, thorough shocking before putting into a −20° C. freezer for 20 min, centrifuging at 15000 rpm for 30 min, collecting 400 μL supernatant for UPLC-Q-TOF-MS analysis.

UPLC conditions: Hypersil GOLD C18 column (100 mm×2.1 mm, 1.9 μm, 190 Å) (Thermo Scientific Co.); mobile phase A: 0.1% formic acid aqueous solution, mobile phase B: 0.1% formic acid acetonitrile; using a gradient elution program from 99% the phase A to 50% the phase A; flow rate 0.4 mL/min; column temperature: 45° C.; and inputting volume: 5 μL.

Q-TOF-MS conditions: a flight time mass analyzer using electrospray ionization (ESI) and a positive ion mode with 200 ng/mL leucine enkephalin for real-time accurate mass calibration; a mass scan range of m/z 80-1000 with a scanning time of 0.3 s; a capillary voltage of 3 kV; a cone voltage of 35V; level-1 mass collision energy of 4; an ion source temperature of 100° C.; a solvent-removing temperature of 300° C. and a solvent-removing gas flow of 500 L/h.

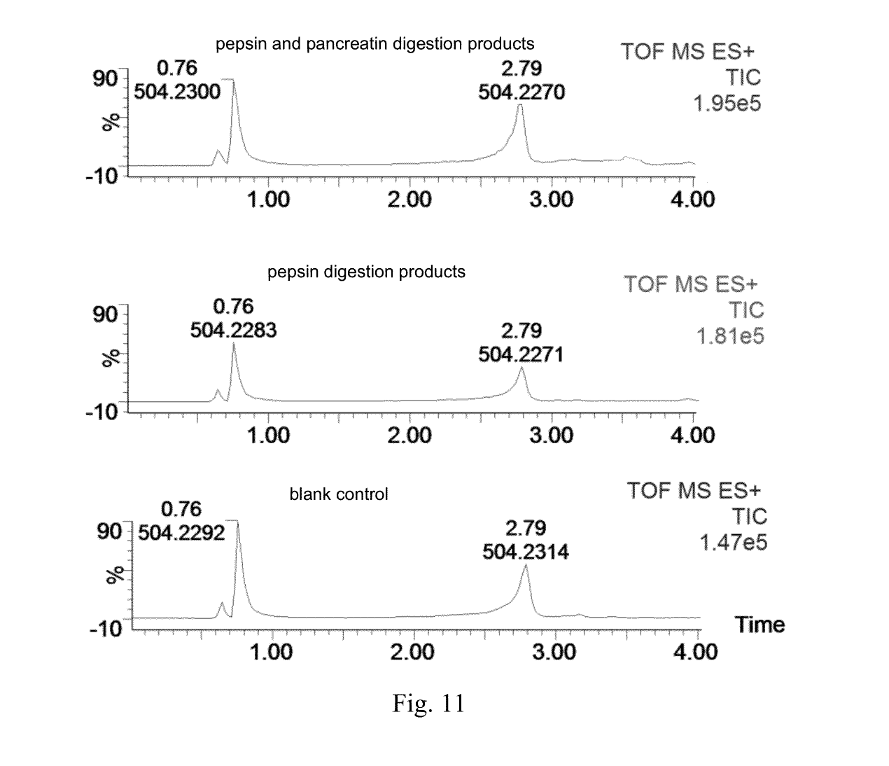

According to the above experiment conditions, the bioactive polypeptides DELQ before and after treatment with digestive enzymes are analyzed by UPLC-Q-TOF-MS, a total ion stream obtained is shown in FIG. 11. Peaks in FIG. 11 are extracted, and corresponding mass chromatography are obtained by Q-TOF-MS analysis.

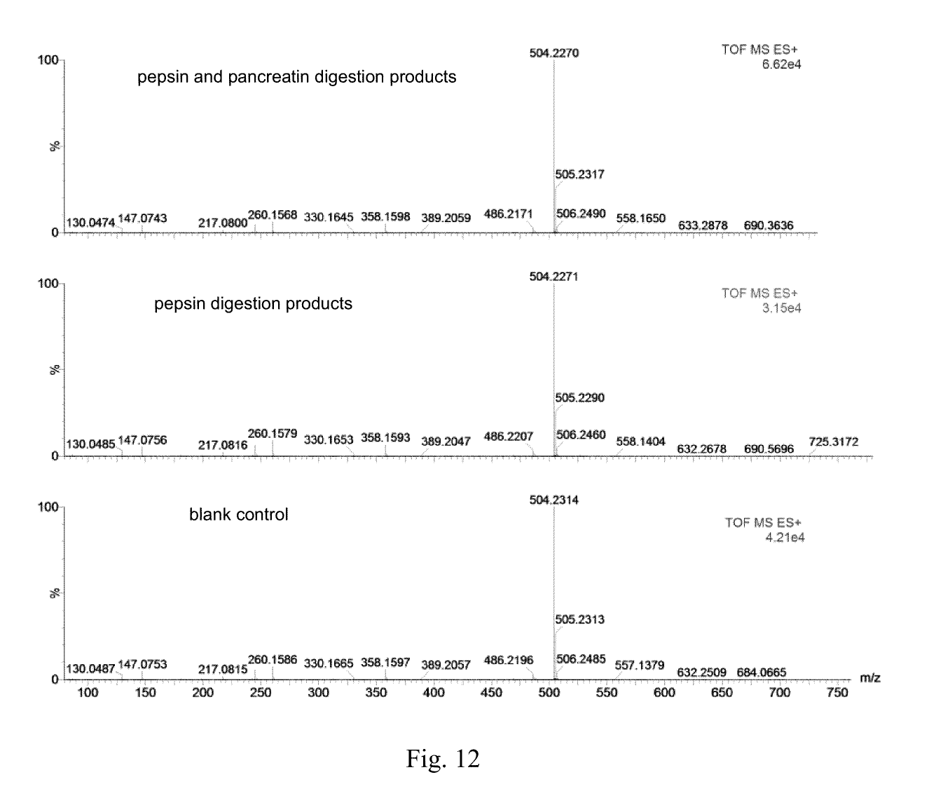

An amino acid sequence of the freeze-dried concentrate of the digestive liquid is detected by the ultra-performance liquid-quadrupole-flight time mass spectrometry. After enzymatic treatment, a level-1 mass chromatography of the DELQ is as shown in FIG. 12, wherein the molecular weight and the amino acid sequence are same with the ones before digestion, which proves that the DELQ is stable under the above condition of simulating in vitro gastrointestinal digestion. The DELQ is not further degraded, and is able to be directly absorbed by the body for playing biological activity.

1-10. (canceled)

11. A bioactive polypeptide, wherein an amino acid sequence thereof is Asp-Glu-Leu-Gln, the bioactive polypeptide is milk-derived.

12. The bioactive polypeptide, as recited in claim 11, wherein a nucleotide fragment thereof is encoded.

13. The bioactive polypeptide, as recited in claim 12, wherein a sequence of the nucleotide fragment is illustrated as SEQ ID NO: 2.

14. A preparation method of a bioactive polypeptide as recited in claim 11, comprising steps of:
1) fermenting: adding CICC6024 Lactobacillus helveticus into skim milk for anaerobic fermentation, in such a manner that Lactobacillus helveticus fermented milk is obtained;
2) coarsely extracting the polypeptide: centrifugally separating the Lactobacillus helveticus fermented milk obtained in the step 1) with a low temperature, and collecting supernatant; and
3) purifying the polypeptide, comprising steps of:
a) ultrafiltering the supernatant obtained in the step 2), and collecting filtrate; and
b) separating the filtrate collected by reversed phase high-performance liquid chromatogram separation through a reversed phase chromatographic column SOURSE 5 RPC ST, and collecting a bioactive polypeptide DELQ.

15. The preparation method, as recited in claim 14, wherein in the step 1), conditions of the anaerobic fermentation are: a fermentation temperature of 36-38° C. and a fermentation time of 15-20 h.

16. The preparation method, as recited in claim 14, wherein in the step a) of the step 3), molecular weight cut-offs of filter membranes for ultrafiltering are respectively 10 kDa and 3 kDa; during ultrafiltering, a pressure range is 0.1-0.3 MPa, and a filtrate flow rate is 0.8-1.2 mL/min.

17. A method for preparing antioxidant and/or immunity-enhancing foods, health products and medicines with a bioactive polypeptide as recited in claim 11, comprising adding an appropriate dose of the polypeptide in the foods, health products and medicines.

18. A medicine with an antioxidant function and an immunity-enhancing function, comprising a bioactive polypeptide DELQ.


SEQUENCE LISTING (3 sequences)

SEQ ID NO: 1 - protein, 4 aa, Artificial sequence
  synthetic bioactive polypeptide
DELQ

SEQ ID NO: 2 - DNA, 12 bp, Artificial sequence
  synthetic bioactive polypeptide coded sequence
gatgaactccag

SEQ ID NO: 3 - protein, 224 aa, Artificial sequence
  synthetic B-Casein amino acid sequence
MKVLILACLVALALARELEELNVPGEIVESLSSSEESITRINKKIEKFQSEEQQQTEDELQDKIHPFAQTQSLVYPFPGPIPNSLPQNIPPLTQTPVVVPPFLQPEVMGVSKVKEAMAPKQKEMPFPKYPVEPFTESQSLTLTDVENLHLPLPLLQSWMHQPHQPLPPTVMFPPQSVLSLSQSKVLPVPQKAVPYPQRDMPIQAFLLYQEPVLGPVRGPFPIIV